(19) United States
(12) Patent Application Publication
(10) Pub. No.: US 2004/0231675 A1
(43) Pub. Date: Nov. 25, 2004

(54) Method and apparatus for transnasal ventilation

(76) Inventor: James R. Lyons, Fairfield, CT (US)

Correspondence Address: Eric R. Moran, McDonnell Boehnen Hulbert & Berghoff, 32nd Floor, 300 S. Wacker Drive, Chicago, IL, 60606, US

(21) Appl. No.: 10/441,557

(22) Filed: May 20, 2003

(51) Int. Cl. A61M 16/00; A62B 7/00

(52) U.S. Cl. 128/207.18; 128/200.26

(57) ABSTRACT

An apparatus and system for delivering oxygen to a nasopharynx and withdrawing exhale gas from the nasopharynx to a carbon dioxide monitor through a tube. In an exemplary embodiment, the tube might be in fluid communication with a junction that can direct oxygen from an oxygen supply through the tube and exhale gas from the tube to the carbon dioxide monitor. Further, the tube might comprise an outer tube and an inner tube.

### BACKGROUND

1. Field of the Invention

The present invention relates to the field of respiratory monitoring of carbon dioxide levels and the supplying of oxygen to a patient.

2. Description of the Related Art

It is often desirable or necessary to exchange gas with a subject, such as a medical patient. Using the example of a medical patient, oxygen can be supplied to the patient, and exhale gases such as carbon dioxide can be collected from the patient. When supplying oxygen to the patient, it may be efficient to transfer oxygen to the patient in a stable and controlled location. Likewise, carbon dioxide levels might be monitored more accurately if based on readings taken at a stable and controlled location. Further, when supplying oxygen to or collecting exhale gases from a patient, errors can occur in the setup of the equipment or apparatus. Therefore, gas supply and/or collection equipment or apparatus that can reduce the risk of error can make gas supply and gas collection safer and more reliable.

Thus, there exists a need for a more stable, more efficient, and safer respiratory monitoring and oxygen supply method and apparatus.

### SUMMARY

In an exemplary embodiment, the transnasal ventilation apparatus can both collect carbon dioxide from a patient's nasopharynx and supply oxygen to a patient's nasopharynx through a tube inserted into the nasopharynx. The tube might fluidly communicate with a junction that can direct exhale gas and oxygen through the tube. More particularly, the junction might direct exhale gas from a patient's nasopharynx to a carbon dioxide monitor and/or the junction might direct oxygen from an oxygen supply to a patient's nasopharynx. In an alternate embodiment, the tube inserted into a patient's nasopharynx might comprise an inner tube and an outer tube. In this embodiment, the inner tube and the outer tube might fluidly communicate with a junction that can direct exhale gas and oxygen through the inner tube and the passageway formed by the inner and outer tubes. More particularly, the junction might direct exhale gas from a patient's nasopharynx to a carbon dioxide monitor and/or the junction might direct oxygen from an oxygen supply to a patient's nasopharynx.

The design of the exemplary embodiments minimize the risk that the apparatus will become dislodged during surgery. In addition, the exemplary embodiments increase safety and control during medical procedures because they maintain oxygen delivery in a more “constant flow” state by supplying constant, passively delivered oxygen to a patient's pharynx. The constant oxygen delivery allows for deeper, more controlled sedation (anesthesia) of the patient. In addition, the exemplary embodiments minimize intrusion on the surgical field of the face.

### BRIEF DESCRIPTION OF THE DRAWINGS

Exemplary embodiments of the present invention are described herein with reference to the drawings, in which:

FIG. 1 is an illustration of an exemplary embodiment;

FIG. 2 is an illustration of a junction shown in FIG. 1;

FIG. 3 is an illustration of another exemplary embodiment;

FIG. 4 is an illustration of another exemplary embodiment; and

FIG. 5 is an illustration of another exemplary embodiment.

### DETAILED DESCRIPTION OF EXEMPLARY EMBODIMENTS

### EXAMPLE 1

1. Overview of Exemplary Embodiments

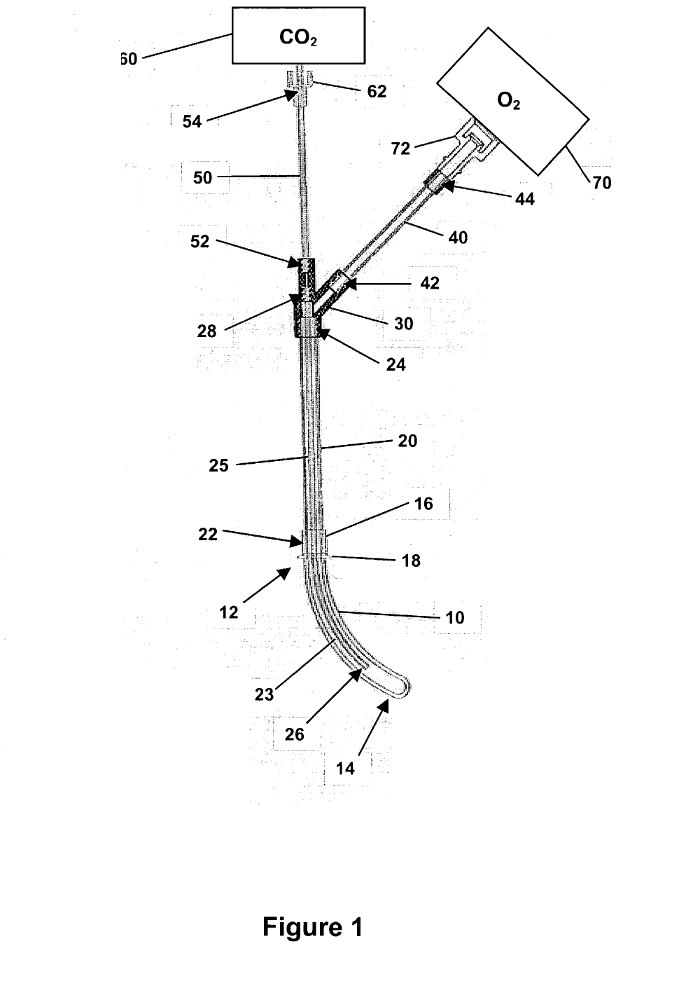

Referring to FIG. 1, in accordance with an exemplary embodiment, a transnasal ventilation apparatus might comprise an insertion guide 10, a first tube 20, a inner tube 25, a junction 30, a second tube 40, and a third tube 50. The first tube 20 might comprise a first end 24 and a second end 22. The inner tube 25 might comprise a first end 26 and a second end 28. The second tube 40 might comprise a first end 42 and a second end 44. And the third tube 50 might comprise a first end 52 and a second end 54. Further, the insertion guide 10, the first tube 20, the inner tube 25, the junction 30, the second tube 40, and the third tube 50 might comprise a single apparatus by, for example, being fused or otherwise bonded together or integral. Other embodiments are possible as well.

Referring to FIG. 1, the insertion guide 10 might comprise a proximal end 12 and a distal end 14. Although it need not be, insertion guide 10 might be tapered. For example, the diameter of the distal end 14 might be larger than the diameter of the proximal end 12. The outside diameters of the proximal end 12 and the distal end 14 may also vary, for example, to accommodate various size nostrils and/or nasal airway passages. The length of the insertion guide 10 may vary as well. In an exemplary embodiment, the distal end 14 of the insertion guide 10 can be inserted into a patient's nasopharynx. In an exemplary embodiment, the insertion guide 10 might be made of a flexible material. For example, the insertion guide 10 might be made of polyvinyl chloride (“PVC”). Other materials, whether flexible or inflexible, are possible as well.

Referring to FIG. 1, in an exemplary embodiment, the proximal end 12 of the insertion guide 10 might comprise a connector 16 and a cuff 18. The connector 16 might receive the first end 22 of the first tube 20. Although not necessary, the connector 16 of the insertion guide 10 might be bonded to the first end 22 of the first tube 20. For example, the connector 16 can be bonded to the first end 22 by an adhesive or through chemical or heat fusing. Other methods of bonding are possible as well. In other embodiments, the connector 16 might be integral with the first end 22. The cuff 18 might contact a patient's nostril and, in addition, might help seal the insertion guide 10 against the patient's nostril.

The first tube 20 might comprise a flexible material, such as PVC. The first tube might also be made of the same material as the insertion guide 10 (which might occur if the insertion guide 10 is integral with or fused to the first tube 20, for instance). Further, the first tube 20 might be made of the same material as the junction 30 (which might occur if the junction 30 is integral with or fused to the first tube 20, for instance). Other examples are possible as well.

In an exemplary embodiment, the first tube 20 might comprise a inner tube 25. For example, the inner tube 25 might be inside the first tube 20 such that the outer surface of the inner tube 25 and the inner surface of the first tube 20 can form a passage 23. The passage 23 might, in turn, provide fluid communication between a patient's air passageways and the junction 30.

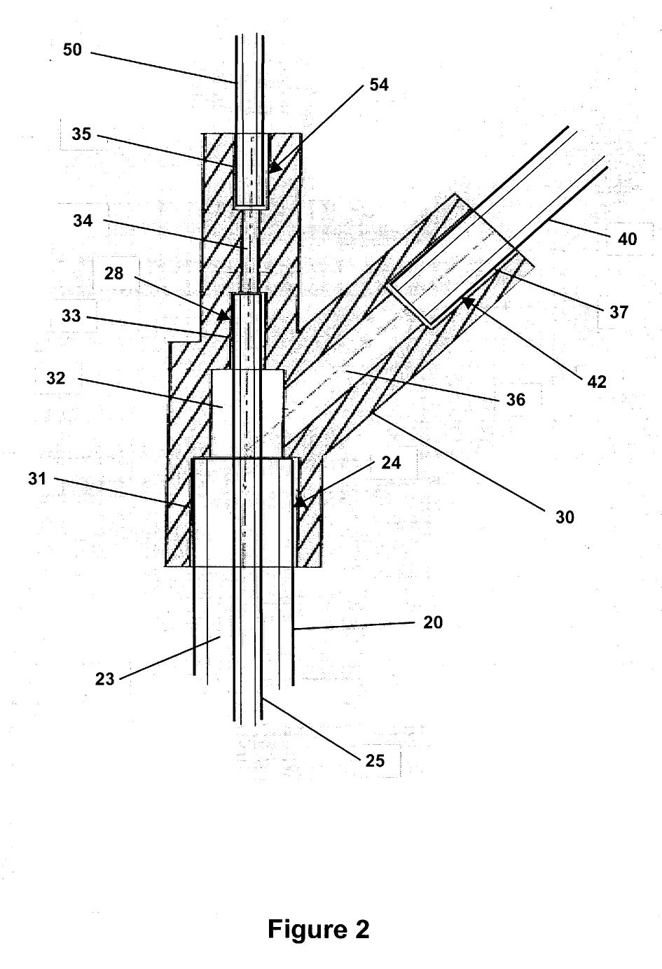

The junction 30 might comprise any type of three-way junction. FIG. 2 depicts an exemplary junction 30 that might comprise seven chambers: a first chamber 31, a second chamber 32, a third chamber 33, a fourth chamber 34, a fifth chamber 35, a sixth chamber 36, and a seventh chamber 37. Other embodiments of junction 30 are possible as well.

In the exemplary embodiments of FIGS. 1 and 2, the first chamber 31 of junction 30 might receive the second end 24 of the first tube 20, and the seventh chamber 37 might receive the first end 42 of the second tube 40. The second chamber 32 and the sixth chamber 36 can then provide fluid communication between the passage 23 and the second tube 40.

Further, the third chamber 33 of junction 30 might receive the second end 28 of the inner tube 25, and the fifth chamber 35 might receive the first end 52 of the third tube 50. The fourth chamber 34 can then provide fluid communication between the inner tube 25 and the third tube 50.

Although not necessary, any combination or all of the first, second, third, or inner tubes 20, 40, 50, and 25 might be bonded to the junction 30. For example, tubes can be bonded to the junction 30 by an adhesive or through chemical or heat fusing. Other methods of bonding are possible as well. In other embodiments, any combination or all of the tubes might be integral with the junction 30.

Other embodiments of the junction 30 and/or the first, second, third, or inner tubes 20, 40, 50, and 25 are possible. For example, portions of the first, second, third, or inner tubes may comprise a single tube. The inner tube 25 and the third tube 50 might comprise a single tube, for instance. In such a case, the third, fourth, and fifth chambers 33, 34, and 35 of junction 30 might comprise a single chamber that can engage the single tube. Other examples are possible as well.

Returning to FIG. 1, the second end 44 of the second tube 40 might be connected to a connector 72. The connector 72 might then connect the second tube 40 to an oxygen supply 70. The second end 54 of the third tube 50 might be connected to a connector 62. The connector 62 might then connect the third tube 50 to a carbon dioxide monitor 60.

The second tube 40 can then fluidly connect the junction 30 to the oxygen supply 70, and the third tube 50 can then fluidly connect the junction 30 to the carbon dioxide monitor 60. The second tube 40 and the third tube 50 might each be made of a flexible material, such as PVC. Other examples are possible as well. For instance, the material of the second tube 40 and the third tube 50 might not be flexible, and the material of any of the first tube 20, the inner tube 25, the second tube 40, or the third tube 50 need not be the same as the material of any other tube. Further, the first, second, third, and inner tubes might also all be made of the same material as the junction 30, which might occur if the first, second, third, or inner tubes are integral with or fused to the junction 30, for instance. The lengths of the first, second, third, and inner tubes might also vary.

2. Exemplary Operation

Referring to FIG. 1, in an exemplary embodiment, a user such as an anesthesiologist (or any other medical or non-medical person) might insert the insertion guide 10 into a patient's nasal passage such that the distal end 14 of the insertion guide 10 extends toward the patient's nasopharynx. In such an arrangement, the proximal end 12 of the insertion guide 10 might frictionally engage the patient's nostril. In an exemplary embodiment, the distal end 14 of the insertion guide 10 might extend beyond the second end 26 of the inner tube 25. In another embodiment, the distal end 14 might not extend beyond the second end 26.

The cuff 18 of the insertion guide 10 might provide a seal around a patient's nostril, thereby providing for more efficient oxygen supply and exhale gas withdrawal. Further, as shown in the embodiment of FIG. 1, the insertion guide 10, the first, second, third, and inner tubes 20, 40, 50, and 25, the junction 30, and connectors 62 and 72 might comprise a single apparatus, thereby providing for quicker assembly and easier use. The single apparatus might also provide for safer use because there are fewer parts to assemble, thereby lowering the risk of improper assembly or other errors.

Referring back to the exemplary embodiment of FIG. 1, the second tube 40 might provide for fluid communication between the junction 30 and an oxygen supply 70. The oxygen supply 70, in turn, might apply a low, positive pressure through the second tube 40, the sixth and second chambers 36 and 32 of junction 30, and the passage 23. The third tube 50 might provide for fluid communication between the junction 30 and a carbon dioxide monitor 60. The carbon dioxide monitor 60, in turn, might apply a low, negative pressure through the third tube 50, the fourth chamber 34 of junction 30, and the inner tube 25.

In accordance with an exemplary embodiment, the transnasal ventilation apparatus can provide for a steady state oxygen supply to/carbon dioxide collection from a patient. As the patient inhales, the patient can draw the lightly pressurized oxygen from the oxygen supply 70 through the passage 23 into the patient's nasopharynx. As the patient exhales, the patient can overcome the supply pressure of the oxygen in the passage 23 and can discharge the exhale gases from the patient's nasopharynx into the inner tube 25. The negative pressure applied by the carbon dioxide monitor 60 can, in turn, withdraw the exhale gases to the carbon dioxide monitor 60.

### EXAMPLE 2

1. Overview of Exemplary Embodiments

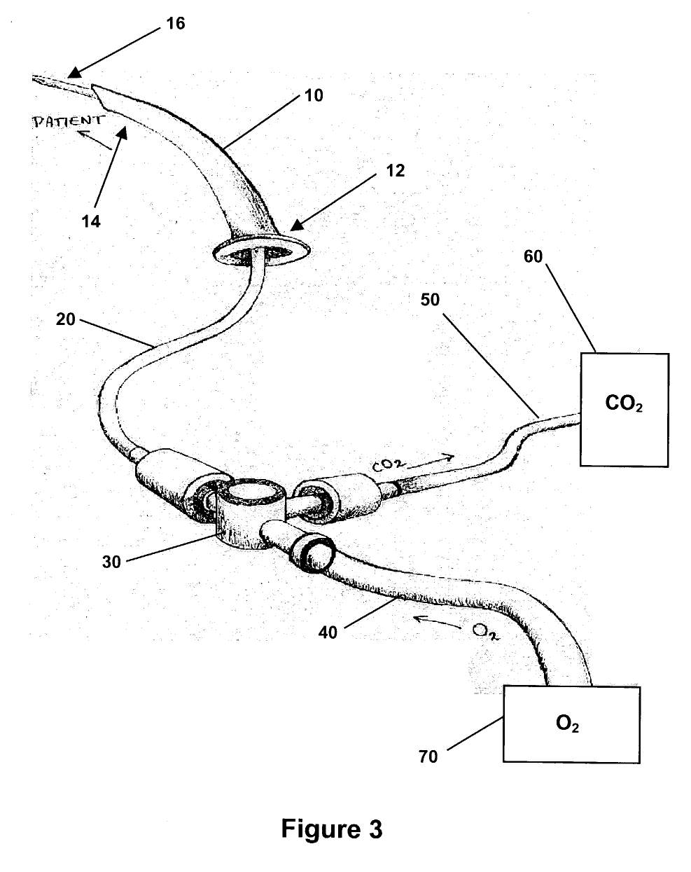

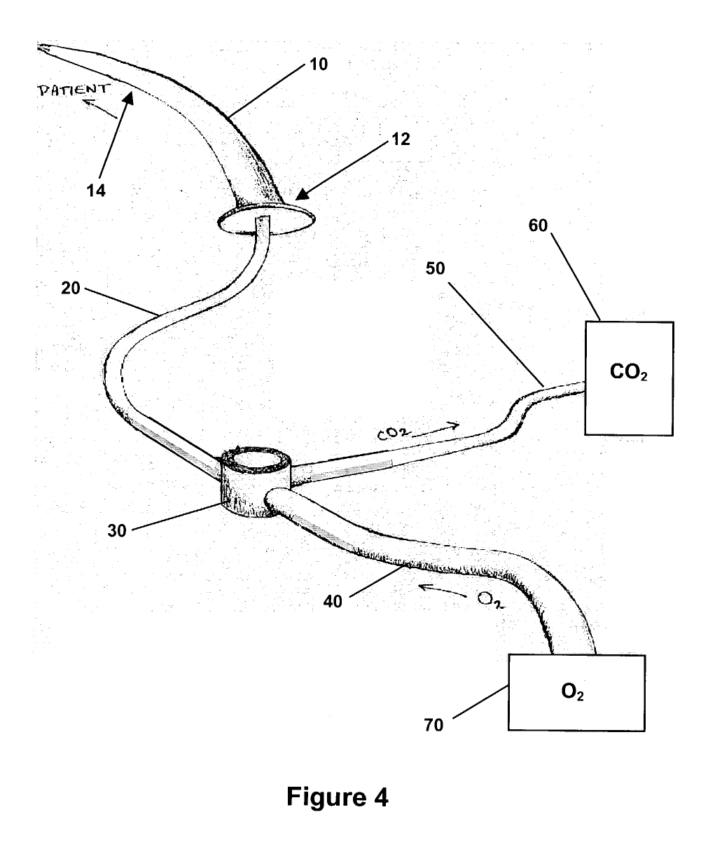

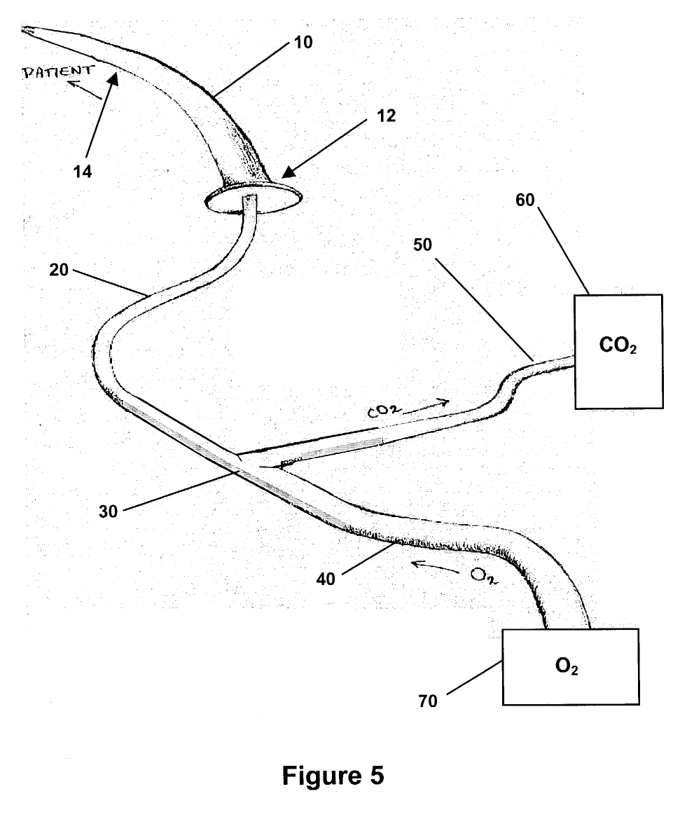

Referring to FIG. 3, in accordance with an exemplary embodiment, a transnasal ventilation apparatus might comprise an insertion guide 10, a first tube 20, a junction 30, a second tube 40, and a third tube 50. Referring to FIG. 4, in accordance with another exemplary embodiment, a transnasal ventilation apparatus might comprise an insertion guide 10, a first tube 20 fixedly attached to the insertion guide 10, a junction 30, a second tube 40, and a third tube 50, the junction 30 being integral with the first, second, and third tubes. FIG. 5 shows an exemplary embodiment similar to the exemplary embodiment of FIG. 4, but with the junction 30 being fused to the first, second, and third tubes. Although not shown, other embodiments are also possible. For instance, in another embodiment, the insertion guide 10 might be fixedly attached to the first tube 20, but the junction 30 might not be integral with or fused to any or all of the first, second, or third tubes. Other examples are possible as well.

Referring to FIGS. 3, 4, and 5, the insertion guide 10 might comprise a proximal end 12 and a distal end 14. Although it need not be, insertion guide 10 might be “bugle” shaped such that the proximal end 12 has a larger circumference than the distal end 14. The outside diameters of the proximal end 12 and the distal end 14 may vary, for example, to accommodate various size nostrils and/or nasal airway passages. In an exemplary embodiment, the outside diameter of the proximal end 12 is 10 mm. In another embodiment, the outside diameter of the proximal end 12 is 8.7 mm. The length of the insertion guide 10 may vary as well.

In an exemplary embodiment, the insertion guide 10 might comprise a cannula. Two examples of commercially available cannulae are the Kendall Argyle™ Nasopharyngeal Airway and the Robertazzi™ Nasopharyngeal Airway. Other examples are possible as well. In an exemplary embodiment, the insertion guide 10 might be made of a flexible material. For example, the insertion guide 10 might be made of rubber latex. As another example, the insertion guide 10 might be made of PVC. Other materials, whether flexible or inflexible, are possible as well.

In an exemplary embodiment, the insertion guide 10 might hold within it a first tube 20. As shown in FIG. 3, for example, the first tube 20 might be slidably inserted into the insertion guide 10. As shown in the embodiments of FIGS. 4 and 5, the first tube 20 might be fixedly attached to the insertion guide 10. For instance, the first tube 20 might be integral with or fused to the insertion guide 10. Other examples are possible as well.

The first tube 20 might comprise a flexible material, such as Silastic™. The first tube might also be made of the same material as the insertion guide 10 (which might occur if the insertion guide 10 is integral with or fused to the first tube 20, for instance). Further, the first tube 20 might be made of the same material as the junction 30 (which might occur if the junction 30 is integral with or fused to the first tube 20, for instance). Other examples are possible as well.

The first tube 20 might, in turn, provide fluid communication between a patient's air passageways and the junction 30. The junction 30 might comprise any type of three-way junction. In one embodiment, the junction 30 might comprise an Airlife™ Tri-Flo® Control Suction Catheter. As shown in the embodiment of FIG. 4, the junction 30 might be integral with the first tube 20, the second tube 40, and the third tube 50. Further, as shown in the embodiment of FIG. 5, the junction 30 might be fused to the first tube 20, the second tube 40, and the third tube 50. Other examples are also possible.

In an exemplary embodiment, the second tube 40 might fluidly connect the junction 30 to an oxygen supply 70, and the third tube 50 might fluidly connect the junction 30 to a carbon dioxide monitor 60. The second tube 40 and the third tube 50 might each be made of a flexible material, such as Silastic™. Other examples are possible as well. For instance, the material of the second tube 40 and the third tube 50 might not be flexible, and the material of any of the first tube 20, the second tube 40, or the third tube 50 need not be the same as the material of any other tube. Further, the first, second, and third tubes might also all be made of the same material as the junction 30, which might occur if the first, second, and third tubes are integral with or fused to the junction 30, for instance. The lengths of the first tube 20, the second tube 40, and the third tube 50 might also vary.

2. Exemplary Operation

Referring to FIG. 3, in an exemplary embodiment, a user such as an anesthesiologist (or any other medical or non-medical person) might insert the insertion guide 10 into a patient's nasal passage such that the distal end 14 of the insertion guide 10 extends toward the patient's nasopharynx. The user can then insert a first, open end 16 of the first tube 20 through the insertion guide 10, such that the first end 16 extends toward the patient's nasopharynx. In such an arrangement, the proximal end 12 of the insertion guide 10 might frictionally engage the patient's nostril. The distal end 14 of the insertion guide 10 might frictionally engage the first end 16 of the first tube 20 and thereby hold the first end 16 in place. In an exemplary embodiment, the insertion guide 10 might hold the first end 16 in place beyond the distal end 14. In another embodiment, the first end 16 might not extend beyond the distal end 14. The first end 16 might also be held in place in other ways as well.

As shown in the embodiments of FIGS. 4 and 5, the insertion guide 10 might be fixedly attached to the first tube 20. The insertion guide 10 might then frictionally engage the nostril and thereby be held in place. In the embodiments of FIGS. 4 and 5, the insertion guide 10 and the integral or fused first tube 20 might provide a seal around a patient's nostril, thereby providing for more efficient oxygen supply and exhale gas withdrawal. Further, as shown in the embodiments of FIGS. 4 and 5, the insertion guide 10 and the first tube 20 might comprise a single component, thereby providing for quicker assembly and easier use. The single insertion guide 10/first tube 20 might also provide for safer use because there are fewer parts to assemble, thereby lowering the risk of improper assembly or other errors.

Referring back to the exemplary embodiments of FIGS. 3, 4, and 5, the second tube 40 might provide for fluid communication between the junction 30 and an oxygen supply 70. The oxygen supply 70, in turn, might apply a low, positive pressure through the second tube 40. The third tube 50 might provide for fluid communication between the junction 30 and a carbon dioxide monitor 60. The carbon dioxide monitor 60, in turn, might apply a low, negative pressure through the third tube 50.

In accordance with an exemplary embodiment, the transnasal ventilation apparatus can provide for a steady state oxygen supply to/carbon dioxide collection from a patient. As the patient inhales, the patient can draw the lightly pressurized oxygen from the oxygen supply 70 through the second tube 40 and through the first tube 20 into the patient's nasopharynx. As the patient exhales, the patient can overcome the supply pressure of the oxygen in the first tube 20 and can discharge the exhale gases from the patient's nasopharynx into the first tube 20. The negative pressure applied by the carbon dioxide monitor 60 can, in turn, withdraw the exhale gases to the carbon dioxide monitor 60.

### CONCLUSION

Several exemplary embodiments of the present invention have been described above. Those skilled in the art will understand, however, that changes and modifications may be made to these embodiments without departing from the true scope and spirit of the present invention, which is defined by the claims.

I claim:

1. A transnasal ventilation apparatus comprising:
a first supply tube and a first exhale tube, each in fluid communication with a fitting member;
a second supply tube and a second exhale tube, the second supply tube in fluid communication with each of the first supply tube and the fitting member, and the second exhale tube in fluid communication with each of the first exhale tube and the fitting member; and
wherein the fitting member allows delivery of oxygen to a nasopharynx through the first supply tube and allows delivery of exhale gases from the nasopharynx through the first exhale tube.

2. The apparatus of claim 1, in combination with an oxygen source and a carbon dioxide monitor, wherein the second supply tube directs oxygen from the oxygen source to the fitting member and the second exhale tube directs carbon dioxide from the fitting member to the carbon dioxide monitor.

3. The apparatus of claim 1, wherein the fitting member, the first supply tube, the first exhale tube, the second supply tube, and the second exhale tube comprise a single apparatus.

4. The apparatus of claim 1, wherein a portion of the first exhale tube is inside a portion of the first supply tube.

5. The apparatus of claim 1, wherein a portion of the first supply tube is inside a portion of the first exhale tube.

6. The apparatus of claim 1, wherein the first exhale tube and the second exhale tube comprise a single exhale tube.

7. The apparatus of claim 1, wherein the first supply tube and the second supply tube comprise a single supply tube.

8. The apparatus of claim 1, wherein the first supply tube comprises an insertion guide.

9. A method comprising:
delivering oxygen from an oxygen source to a nasopharynx through a first supply tube and delivering exhale gas from the nasopharynx to a carbon dioxide monitor through a first exhale tube, wherein the first supply tube is in fluid communication with a fitting member and a second supply tube, and the first exhale tube is in fluid communication with the fitting member and a second exhale tube.

10. The method of claim 9, wherein the second supply tube directs oxygen from the oxygen source to the fitting member and the second exhale tube directs carbon dioxide from the fitting member to the carbon dioxide monitor.

11. The method of claim 9, wherein at least a portion of the first exhale tube is inside at least a portion of the first supply tube.

12. The method of claim 9, wherein at least a portion of the first supply tube is inside at least a portion of the first exhale tube.

13. The apparatus of claim 9, wherein the first exhale tube and the second exhale tube comprise a single exhale tube.

14. The apparatus of claim 9, wherein the first supply tube and the second supply tube comprise a single supply tube.

15. The apparatus of claim 9, wherein the fitting member, the first supply tube, the first exhale tube, the second supply tube, and the second exhale tube comprise a single apparatus.

16. A transnasal ventilation apparatus comprising:
a first tube in fluid communication with a fitting member;
a second tube and a third tube, each in fluid communication with the first tube and the fitting member; and
wherein the fitting member allows delivery of oxygen to a nasopharynx through the first tube and allows delivery of exhale gases from the nasopharynx through the first tube.

17. The apparatus of claim 16, in combination with an oxygen source and a carbon dioxide monitor, wherein the second tube directs oxygen from the oxygen source to the fitting member and the third tube directs carbon dioxide from the fitting member to the carbon dioxide monitor.

18. The apparatus of claim 16, wherein the fitting member, the first tube, the second tube, and the third tube comprise a single apparatus.

19. The apparatus of claim 16, wherein the first tube comprises an inner tube, and wherein the first tube and the inner tube comprise a passage.

20. The apparatus of claim 19, wherein the passage is in fluid communication with the second tube and the inner tube is in fluid communication with the third tube.

21. The apparatus of claim 20, wherein the inner tube and the third tube comprise a single tube.

22. The apparatus of claim 19, wherein the fitting member, the first tube, the inner tube, the second tube, and the third tube comprise a single apparatus.

23. The apparatus of claim 16, wherein the fitting member, the first tube, the second tube, and the third tube comprise a single apparatus.

24. A method comprising:
delivering oxygen from an oxygen source to a nasopharynx and delivering exhale gas from the nasopharynx to a carbon dioxide monitor through a first tube, wherein the first tube is in fluid communication with a fitting member, a second tube, and a third tube.

25. The method of claim 24, wherein the second tube directs oxygen from the oxygen source to the fitting member and the third tube directs carbon dioxide from the fitting member to the carbon dioxide monitor.

26. The method of claim 24, wherein the first tube comprises an inner tube, and the first tube and the inner tube comprise a passage, wherein the passage is in fluid communication with the second tube and the inner tube is in fluid communication with the third tube.

27. The method of claim 26, wherein the fitting member, the first tube, the inner tube, the second tube, and the third tube comprise a single apparatus.

28. The apparatus of claim 26, wherein the inner tube and the third tube comprise a single tube.

29. A transnasal ventilation apparatus comprising:
a first supply tube in fluid communication with a second supply tube; and
a first exhale tube in fluid communication with a second exhale tube;
wherein the first and second supply tubes allow delivery of oxygen to a nasopharynx and the first and second exhale tubes allow delivery of exhale gases from the nasopharynx.

30. The apparatus of claim 29, wherein at least a portion of the first exhale tube is inside at least a portion of the first supply tube.

31. The apparatus of claim 29, wherein at least a portion of the first supply tube is inside at least a portion of the first exhale tube.

32. The apparatus of claim 29, wherein the first and second supply tubes comprise a single supply tube.

33. The apparatus of claim 32, in combination with an oxygen source, wherein the single supply tube directs oxygen from the oxygen source to the nasopharynx.

34. The apparatus of claim 29, wherein the first and second exhale tubes comprise a single exhale tube.

35. The apparatus of claim 34, in combination with a carbon dioxide monitor, wherein the single exhale tube directs carbon dioxide from the nasopharynx to the carbon dioxide monitor.

36. The apparatus of claim 29, further comprising a fitting member in fluid communication with the first and second supply tubes.

37. The apparatus of claim 36, wherein the first and second exhale tubes comprise a single exhale tube.

38. The apparatus of claim 29, further comprising a fitting member in fluid communication with the first and second exhale tubes.

39. The apparatus of claim 38, wherein the first and second supply tubes comprise a single supply tube.

40. The apparatus of claim 29, further comprising a fitting member in fluid communication with the first and second supply tubes and in fluid communication with the first and second exhale tubes.

41. The apparatus of claim 40, in combination with an oxygen source and a carbon dioxide monitor, wherein the second supply tube directs oxygen from the oxygen source to the fitting member and the second exhale tube directs carbon dioxide from the fitting member to the carbon dioxide monitor.
